(19) United States
(12) Patent Application Publication
(10) Pub. No.: US 2026/0306494 A1
(43) Pub. Date: Oct. 8, 2026

(54) LIQUID AND SOLID COMPOSITIONS OF IMIPRIDONE DERIVATIVES

(71) Applicants: INSTITUT GUSTAVE ROUSSY, VILLEJUIF (FR); UNIVERSITE PARIS-SACLAY, GIF SUR YVETTE (FR); ASSISTANCE PUBLIQUE - HÔPITAUX DE PARIS, PARIS (FR)

(72) Inventors: MAXIME ANNEREAU, VINCENNES (FR); BERNARD DO, ISSY LES MOULINEAUX (FR); ANDRÉ RIEUTORD, PARIS (FR); PHILIPPE-HENRI SECRETAN, VIROFLAY (FR); MARINA VIGNES, PARIS (FR)

(21) Appl. No.: 19/476,746

(22) PCT Filed: Apr. 19, 2024
(86) PCT No.: PCT/EP2024/060671

§ 371 Date: Oct. 20, 2025

(30) Foreign Application Priority Data
Apr. 20, 2023 (EP) 23305612.6

(51) Int. Cl. A61K 31/519; A61K 9/00; A61K 9/08; A61K 31/455; A61K 47/26; A61K 47/38; A61K 47/46

(52) U.S. Cl. CPC A61K 31/519; A61K 9/0056; A61K 9/006; A61K 9/08; A61K 31/455; A61K 47/26; A61K 47/38; A61K 47/46

(57) ABSTRACT

A liquid pharmaceutical composition comprising an aqueous solvent, nicotinamide and at least one imipridone derivative of formula (I). A process for manufacturing said liquid pharmaceutical composition, wherein the process comprises the steps of (a) Dissolving nicotinamide in an aqueous solvent, preferably water; (b) Adding at least one pH modifier and at least one imipridone derivative to the solution obtained in step (a); and (c) Optionally sterilizing the solution obtained in step (b). A 1:1 imipridone derivative nicotinamide complex or cocrystal, wherein the imipridone derivative is of formula (I). Said compositions for use in the treatment of cancer.

### TECHNICAL FIELD

The invention relates to liquid and solid pharmaceutical compositions of imipridone derivatives, and their use in the treatment of cancer, especially of glioma.

### TECHNICAL BACKGROUND

H3K28 altered diffuse midline glioma (DMG) are among the most challenging tumors to treat both in children and adults, with limited therapeutic options and a very poor prognosis that has not changed during the last decades. In this context, new therapeutic options are urgently needed to improve the treatment of this disease, but specific clinical trials did not identify new efficient drugs. After some encouraging signals from a phase II study and a window-opportunity-arm, imipridone ONC201 has emerged as an interesting compound after some patients with recurrent H3K28M DMG have shown a sustained response.

Unfortunately, imipridone presents stability issues both in free form and as a salt. First, in its non-protonated form, imipridone is not soluble in water. When a salt thereof, such as a hydrochloride salt thereof, is used, imipridone is solubilized in water. For instance, ONC201.2HCl is soluble at a rate of 41.4 g/L (ie 4.1% w/v) in water. However, the formation of hydrates of imipridone inorganic salts, such as hydrochloride, even at concentrations as low as 40 g/L in water, reduces its solubility in water over time, thus triggering a progressive loss in stability of the imipridone inorganic salt liquid composition. In addition, imipridone is sensitive to light and to oxidation.

It is thus difficult to develop liquid pharmaceutical compositions of imipridone or derivatives thereof, comprising sufficient amounts of solubilized imipridone or derivatives thereof to obtain a therapeutical effect in a patient, and which remain stable over time.

In addition, oral formulations such as liquid oral formulations are highly desirable for active agents, especially for active agents which are to be administered to children and/or to people with swallowing difficulties. The use of oral formulations, such as liquid oral formulations, is suitable for improving the patient compliance. Furthermore, liquid aqueous formulations of solubilized active agents and solid formulations obtained therefrom provide the active agent to the patient in a form that is bioavailable for the patient, thus improving its therapeutical effect.

Thus, there remains a need to provide novel liquid formulations of imipridone derivatives, which would comprise high enough amounts of imipridone derivatives to obtain a therapeutical effect in a patient, said imipridone derivatives being preferably in a bioavailable form, and which remain stable over time.

### SUMMARY OF THE INVENTION

In this respect, the inventors have surprisingly demonstrated that the use of nicotinamide allows drastically increasing the solubility of imipridone derivatives in aqueous solvents, and improves the stability of aqueous solutions of imipridone derivatives over time.

Thus, the present invention relates to a liquid pharmaceutical composition comprising an aqueous solvent, nicotinamide and at least one imipridone derivative of formula (I):

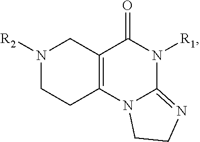

wherein R₁ is an alkyl group substituted by a heterocycle or a substituted benzyl group, and R₂ is a hydrogen group or a benzyl group, or a pharmaceutically acceptable salt and/or solvate thereof.

In some embodiments, the imipridone derivative of formula (I) is selected from the group consisting of ONC201, ONC206 and ONC212, preferably it is ONC201 or a pharmaceutically acceptable salt and/or solvate thereof, more preferably it is ONC201.2HCl.

In some embodiments, the aqueous solvent is water or a 1:1 w:w water:ethanol mixture. In some embodiments, the composition further comprises at least one pharmaceutically acceptable pH modifier.

In some embodiments, the composition further comprises at least one of an isotonizing agent, a thickening and/or gelling agent, a preservative, such as an antimicrobial agent and/or an antioxidant agent, a flavoring agent, a sweetening agent, and/or a surfactant, preferably a non-ionic surfactant.

The isotonizing agent may be selected from the group consisting of a polyol, a sugar and a linear or cyclic glucitol having from 2 to 10 carbon atoms, for instance selected from mannitol, sorbitol, inositol, glucose and glycerol.

The thickening and/or gelling agent may be selected from the group consisting of polyvinyl alcohol, gelatin, cellulose derivatives, such as sodium carboxymethyl cellulose or hypromellose, povidone derivatives such as polyvinyl pyrrolidone; copovidone or povidone acetate, alginate derivatives, starch such as corn starch and gums such as guar gum, xanthan gum and/or carrageenan gum.

The antibacterial agent may be selected from the group consisting of sorbic acids, potassium sorbate, benzoates such as sodium benzoate, and parabens (alkyl parahydroxybenzoates).

The antioxidant agent may be selected from the group consisting of ascorbic acid, erythorbic acid, monothioglycerol, sodium bisulfite, sodium metabisulfite, N-acetylcysteine, and a reducing sugar.

The sweetening agent may be selected from the group consisting of sucrose, sucralose, trehalose, saccharine, sodium saccharine, aspartame, acesulfame, stevia, maltodextrin and neohesperidin dihydrochalcone.

The flavoring agent may be selected from the group consisting of menthol, citrus, cola, strawberry, orange, mint and pepper mint.

The surfactant may be selected from the group consisting of polyoxyethylene hydrogenated castor oil, polysorbate 20 and 80 and caprylocaproyl polyoxylglycerides.

In some embodiments, the weight ratio of imipridone derivative to nicotinamide in the liquid pharmaceutical composition is comprised between 0.5 and 2, preferably between 1 and 1.2. In some embodiments, the amount of imipridone derivative is between 20 mg/mL and 420 mg/mL, preferably between 30 mg/mL and 200 mg/mL, more preferably between 80 and 150 mg/mL, in particular about 100 mg/mL.

In some embodiments, the amount of nicotinamide is between 20 mg/mL and 420 mg/mL, preferably between 30 mg/mL and 200 mg/mL, more preferably between 80 and 150 mg/mL, in particular about 100 mg/mL.

The present invention also relates to a process for manufacturing a liquid pharmaceutical composition according to the invention, comprising the steps of:
(a) Dissolving nicotinamide in an aqueous solvent, preferably water;
(b) Adding at least one pH modifier and at least one imipridone derivative to the solution obtained in step (a); and
(c) Optionally sterilizing the solution obtained in step (b).

The present invention also relates to a solid pharmaceutical composition comprising at least one imipridone derivative, or a pharmaceutically acceptable salt and/or solvate thereof and nicotinamide, which is obtained from a liquid aqueous composition comprising an aqueous solvent, the at least one imipridone derivative, or a pharmaceutically acceptable salt and/or solvate thereof and nicotinamide.

The form of the solid pharmaceutical composition may be selected from the group consisting of gummies, dragees, powders for reconstitution in water, orodispersible films, hard-gel capsules and soft-gel capsules.

The present invention also relates to a 1:1 imipridone derivative nicotinamide complex or cocrystal, wherein the imipridone derivative is preferably ONC201, more preferably ONC201.2HCl.

The present invention also relates to a pharmaceutical composition comprising nicotinamide and an imipridone derivative of formula (I)

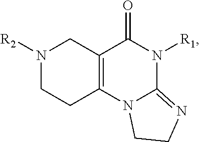

wherein R₁ is an alkyl group substituted by a heterocycle or a substituted benzyl group and R₂ is a hydrogen atom or a benzyl group, or a pharmaceutically acceptable salt and/or solvate thereof, for use as a medicament.

The present invention also relates to a pharmaceutical composition comprising nicotinamide and an imipridone derivative of formula (I)

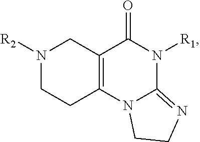

wherein R₁ is an alkyl group substituted by a heterocycle or a substituted benzyl group and R₂ is a hydrogen atom or a benzyl group, or a pharmaceutically acceptable salt and/or solvate thereof, for use in the treatment of a cancer.

The cancer may be selected from the group consisting of prostate carcinoma, pancreatic cancer, lung carcinoma, breast cancer, glioma, cancers of head and neck, colon cancer and skin cancer, preferably it is a glioma, more preferably a diffuse midline glioma.

In some embodiments, the pharmaceutical composition is a liquid pharmaceutical composition according to the invention, or a solid pharmaceutical composition according to the invention.

### BRIEF DESCRIPTION OF FIGURES

FIG. 1 presents photographs of the solution of ONC201.2HCl in absence of nicotinamide of example 4 after 3 days of storage at room temperature (a), and at the beginning of the experiment (b).

FIG. 2 presents a microscopy image of the mixture of FIG. 1 (a), observed at 20× magnification.

FIG. 3 presents a photograph of the solution of ONC201.2HCl in presence of nicotinamide of example 4 after 5 days of storage at room temperature (a) and a microscopy image of the solution, observed at 20× magnification (b).

FIG. 4 is a graph showing evolution of the amount of imipridone derivative over time in the solution of example 5.

FIG. 5 presents the infrared spectra of (a) a liquid composition comprising a 1:1 complex of nicotinamide and ONC201.2HCl, (b) nicotinamide alone and (c) ONC201.2HCl alone.

### DETAILED DESCRIPTION OF THE INVENTION

### Definitions

According to the present invention, an “alkyl” group is a monovalent linear or branched saturated hydrocarbon chain. An alkyl group preferably comprises from 1 to 10 carbon atoms, more preferably from 1 to 6 carbon atoms, in particular from 1 to 3 carbon atoms. For example, an alkyl more specifically means methyl, ethyl, n-propyl, isopropyl, n-butyl, iso-butyl, sec-butyl, tert-butyl, pentyl or linear or branched hexyl.

An “aqueous solvent” is a solvent comprising water, and optionally at least another solvent, which is preferably water-miscible.

The term “about” is used herein to mean approximately, roughly, around, or in the region of. When the term “about” is used in conjunction with a numerical range, it modifies that range by extending the boundaries above and below the numerical value of plus and minus 15% the numerical value, preferably between plus and minus 10%, more preferably between plus and minus 5%.

The term “halogen” or “halo” refers to a fluorine, bromine, chlorine or iodine atom.

A “heterocycle” is an organic compound comprising a closed chain of atoms, at least one of which is not a carbon atom (a heteroatom). Each heteroatom may be independently a N, O or S atom.

An “imipridone derivative” is a compound of general formula (I):

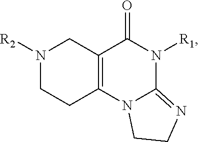

wherein R₁ is an alkyl group substituted by a heterocycle or a substituted benzyl group and R₂ is a hydrogen atom or a benzyl group. If not otherwise specified, the term “imipridone derivative” also encompasses pharmaceutical salts and/or solvates of the compound of formula (I) above. The term “imipridone derivative” may also encompass stereoisomers and/or tautomers of the compound of formula (I) if applicable.

An “isotonizing agent” or “isotonic agent” is an agent intended to create an osmotic pressure in the region of that of physiological saline.

An “oral” composition is a composition that is suitable for an administration to a patient via the oral, buccal and/or transmucosal route.

A “pH modifier” is a compound, preferably acidic or basic, which contributes to obtaining the desired pH for a composition.

“Pharmaceutically acceptable” refers to what is useful to the preparation of a pharmaceutical composition, and what is generally safe and non-toxic, for a pharmaceutical use.

A “pharmaceutically acceptable salt” is a salt of a compound which is pharmaceutically acceptable, as defined above, and which possesses the pharmacological activity of the corresponding compound. The pharmaceutically acceptable salts comprise:
(1) acid addition salts formed with inorganic acids such as hydrochloric, hydrobromic, sulfuric, nitric and phosphoric acid and the like; or formed with organic acids such as acetic, benzenesulfonic, fumaric, glucoheptonic, gluconic, glutamic, glycolic, hydroxynaphthoic, 2-hydroxyethanesulfonic, lactic, maleic, malic, mandelic, methanesulfonic, muconic, 2-naphtalenesulfonic, propionic, succinic, dibenzoyl-L-tartaric, tartaric, p-toluenesulfonic, trimethylacetic, and trifluoroacetic acid and the like, and
(2) salts formed when an acid proton present in the compound is either replaced by a metal ion, such as an alkali metal ion, an alkaline-earth metal ion, or an aluminum ion; or coordinated with an organic or inorganic base. Acceptable organic bases comprise diethanolamine, ethanolamine. N-methylglucamine, triethanolamine, tromethamine and the like. Acceptable inorganic bases comprise aluminum hydroxide, calcium hydroxide, potassium hydroxide, sodium carbonate and sodium hydroxide.

A “solvate” of a compound is a molecular complex comprising the compound and one or more pharmaceutically acceptable solvent molecules, for example, water or ethanol. When the solvent is water, the solvate is called a hydrate.

A liquid solution containing an active agent is “stable” for a certain period of time if, during this period, the physico-chemical properties and microbial quality of the medicinal product remain within the limits that ensure its continued efficacy and safety. Preferably, over this period of time, the concentration of active agent in the solution (dissolved active agent) does not decrease of more than 10%, preferably no more than 5%, and/or the formation of degradation products follows the requirements of the ICH Q3 guidelines (ICH refers to International Council for Harmonisation of Technical Requirements for Pharmaceuticals for Human Use). A liquid solution comprising less than 0.5% of degradation products is compliant with said guidelines.

“Stereoisomers” are isomeric compounds that have the same molecular formula and sequence of bonded atoms, but differ in the 3D-dimensional orientations of their atoms in space. The stereoisomers include enantiomers, diastereoisomers, cis-trans and E-Z isomers, conformers, and anomers. In a specific aspect of the disclosure, the stereoisomers include diastereoisomers and enantiomers.

A “subject” is an animal, including a human. In the sense of the present invention, a subject may be a patient, i.e. a person receiving medical attention, undergoing or having underwent a medical treatment, or monitored for the development of a disease. In one embodiment, the subject is an adult (for example a subject above the age of 18). In another embodiment, the subject is a child (for example a subject below the age of 18). In one embodiment, the subject is a male. In another embodiment, the subject is a female.

“Tautomers” are isomeric compounds that differ only in the position of the protons and the electrons.

A “therapeutically effective amount” is the amount of a therapeutic agent necessary and sufficient for slowing down or stopping the progression, aggravation, or deterioration of one or more symptoms of the disease, or condition; alleviating the symptoms of the disease or condition, and/or curing the disease or condition.

“Transmucosal administration” refers to the administration of a product through or across a mucosal tissue. In the meaning of this invention, preferred mucosal tissues are buccal, sublingual, gingival and palatal tissues. According to the invention, the transmucosal administration is preferably buccal administration. In this case, the administered substance reaches the systemic circulation through or across the mucosal tissues of the buccal cavity but is not ingested or swallowed.

“Treating” or “treatment” refers to both therapeutic treatment and prophylactic or preventative measures; wherein the object is to prevent or slow down (lessen) the targeted pathologic condition or disorder. Those in need of treatment include those already with the disorder as well as those prone to have the disorder or those in whom the disorder is to be prevented. A subject or mammal is successfully “treated” for an infection if, after receiving a therapeutic amount of an antibody according to the methods of the present invention, the patient shows observable and/or measurable reduction in or absence of one or more parameters, such as the following: reduction in the number of pathogenic cells; reduction in the percent of total cells that are pathogenic; and/or relief to some extent, one or more of the symptoms associated with the specific disease or condition; reduced morbidity and mortality, and improvement in quality of life issues. The above parameters for assessing successful treatment and improvement in the disease are readily measurable by routine procedures familiar to a physician.

### Liquid Composition

A first object of the present invention is a liquid pharmaceutical composition comprising an aqueous solvent, nicotinamide and at least one imipridone derivative, or a pharmaceutically acceptable salt and/or solvate thereof.

The presence of nicotinamide in the liquid composition according to the invention allows dissolving a higher concentration of imipridone derivative in the aqueous solvent. The concentration of imipridone derivative dissolved in the aqueous solvent may range up to 200 mg/mL at room temperature, that is about 5 times higher than intrinsic solubility thereof in water.

Without wishing to be bound by a theory, the Inventors believe that the solubility increase of imipridone derivatives and pharmaceutically acceptable salts thereof in aqueous solvents in presence of nicotinamide may be due to a complexation between the imipridone skeleton and nicotinamide, due to the formation of hydrogen bonds and/or of dipole-dipole interactions between both molecules.

The solubility increase of the imipridone derivatives and pharmaceutically acceptable salts thereof is additionally believed to improve its resistance to oxidation, as the amount of dissolved oxygen molecules will be lower, due to a salting-out phenomenon.

Consequently, the presence of nicotinamide allows increasing the amount of solubilized imipridone derivative in aqueous solvent, improving the stability of aqueous solution of imipridone derivatives, and improving the resistance to oxidation of imipridone derivatives.

The liquid pharmaceutical composition according to the invention is stable against degradation and/or flocculation and/or precipitation and/or re-crystallization of the active ingredients, in particular of the at least one imipridone derivative, especially under prolonged storage. The prolonged storage may be at low temperature, but also at room temperature. During the prolonged storage, the liquid pharmaceutical composition may be protected from light.

The imipridone derivative is a compound of formula (I)

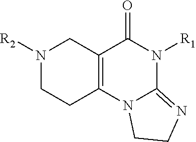

wherein R₁ is an alkyl group substituted by a heterocycle or a substituted benzyl group and R₂ is a hydrogen atom or a benzyl group, or a pharmaceutically acceptable salt and/or solvate thereof.

When R₁ is an alkyl group substituted by a heterocycle, the alkyl group is preferably a methyl group or an ethyl group, more preferably a methyl group. The heterocycle is preferably selected from the group consisting of thienyl, isoxazolidine, morpholine and pyridine.

When R1 is a substituted benzyl group, the benzyl group of R₁ may be substituted by at least one substituent, preferably substituted by one or two substituents. Each substituent may be independently selected from the group consisting of an alkyl group, preferably a methyl group, an alkyloxy group, such as a methoxy group, a halogen atom, preferably a chlorine or a fluorine atom, and a halogenoalkyl group, such as a trifluoromethyl group. In a preferred embodiment, each substituent is selected from the group consisting of a methyl group, a fluorine atom and a trifluoromethyl group.

In a preferred embodiment, R₁ is a substituted benzyl group. In a preferred embodiment, R₂ is a benzyl group. In a highly preferred embodiment, R₁ is a substituted benzyl group and R₂ is a benzyl group.

Imipridone derivatives of formula (I) may be selected from the group consisting of ONC201, ONC203, ONC206, ONC207, ONC210, ONC211, ONC212, ONC213, ONC217, ONC218, ONC219, ONC220, ONC221, ONC222, ONC223, ONC224, ONC225 and ONC226. The structures of these compounds are known in the art as follows (see Jessica Wagner et al., CELL CYCLE 2017, Vol. 16, No. 19, 1790-1799):

| Imipridone | R₂ | R₁ |
|---|---|---|
| ONC201 | CH₂Ph | CH₂—((2—CH₃)—Ph) |
| ONC203 | CH₂Ph | CH₂—(thienyl) |
| ONC206 | CH₂Ph | CH₂—(2,4-di F—Ph) |
| ONC207 | H | CH₂—((2—CH₃)—Ph) |
| ONC210 | CH₂Ph | CH₂—(3,5-di F—Ph) |
| INC211 | CH₂Ph | CH₂—(3,4-di Cl—Ph) |
| ONC212 | CH₂Ph | CH₂—(4—CF₃—Ph) |
| ONC213 | CH₂Ph | CH₂—(3,4-di F—Ph) |
| ONC217 | CH₂Ph | CH₂—(2—F—Ph) |
| ONC218 | CH₂Ph | CH₂—(2—CH₃, 4-F—Ph) |
| ONC219 | CH₂Ph | CH₂—(2,4-di Cl—Ph) |
| ONC220 | CH₂Ph | CH₂—((4—OCH₃)—Ph) |
| ONC221 | H | CH₂—(4—CF₃—Ph) |
| ONC222 | CH₂Ph | CH₂—(3-isoxazolidinyl) |
| ONC223 | CH₂Ph | CH₂—(4CH₃—Ph) |
| ONC224 | CH₂Ph | CH₂CH₂—(4-morpholinyl) |
| ONC225 | CH₂Ph | CH₂—(2—F, 4—CF₃—Ph) |
| ONC226 | CH₂Ph | CH₂—(3-pyridinyl) |

The imipridone derivative is preferably selected from the group consisting of:
ONC201 (7-Benzyl-4-(2-methylbenzyl)-1,2,6,7,8,9-hexahydroimidazo[1,2-a]pyrido[3,4-e]pyrimidin-5 (4H)-one),
ONC206 (7-Benzyl-4-(2,4-difluorobenzyl)-1,2,6,7,8,9-hexahydroimidazo[1,2-a]pyrido[3,4-e]pyrimidin-5 (4H)-one),
ONC212 (7-Benzyl-4-(4-(trifluoromethyl)benzyl)-1,2,6,7,8,9-hexahydroimidazo[1,2-a]pyrido[3,4-e]pyrimidin-5 (4H)-one),
pharmaceutically acceptable salts and/or solvates thereof, and mixtures thereof.

The chemical formulae of ONC201, ONC206 and ONC212 are reproduced below.

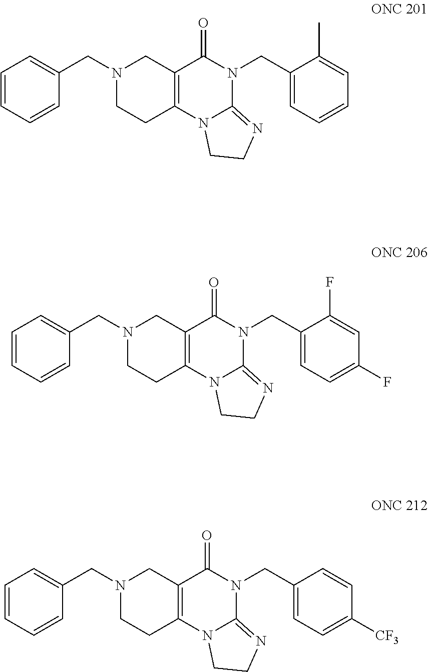 [SMILES: Cc1ccccc1CN1C(=O)C2=C(CCN(Cc3ccccc3)C2)N2CCN=C12 ; O=C1C2=C(CCN(Cc3ccccc3)C2)N2CCN=C2N1Cc1ccc(F)cc1F ; O=C1C2=C(CCN(Cc3ccccc3)C2)N2CCN=C2N1Cc1ccc(C(F)(F)F)cc1]

In a preferred embodiment, the imipridone derivative is ONC201, or a pharmaceutically acceptable salt and/or solvate thereof, in particular ONC201.2HCl.

Nicotinamide, also known as niacinamide, is a derivative of nicotinic acid. Nicotinamide is a water-soluble vitamin and is part of the B group of vitamins. Nicotinamide is pharmaceutically acceptable. In the present invention, nicotinamide is present in the pharmaceutical compositions in an amount that is lower than its NOAEL (No Observed Adverse Effect Level), which is known to be around 200 mg/kg bw (body weight). The presence of nicotinamide does thus not affect the therapeutical effects of the active agent(s) present in the compositions.

The amount of imipridone derivative in the liquid pharmaceutical composition may vary in a wide range. It is preferably between 20 mg/mL and 420 mg/mL, preferably between 30 mg/mL and 200 mg/mL, more preferably between 80 and 150 mg/mL, in particular about 100 mg/mL.

The amount of nicotinamide in the liquid pharmaceutical composition may vary in a wide range. It is preferably between 20 mg/mL and 420 mg/mL, preferably between 30 mg/mL and 200 mg/mL, more preferably between 80 and 150 mg/mL, in particular about 100 mg/mL.

The weight ratio between the imipridone derivative and nicotinamide in the liquid pharmaceutical composition may vary in a wide range. Preferably, the weight ratio of imipridone derivative to nicotinamide is comprised between 0.5 and 2, preferably between 1 and 1.2. The molar ratio of imipridone derivative to nicotinamide is preferably comprised between 0.2 to 0.6, in particular it is about 0.42. In some other embodiments, the molar ratio of imipridone derivative to nicotinamide may be comprised between 0.98 and 1.02, in particular it is about 1.

In a specific embodiment, the liquid pharmaceutical composition comprises about 100 mg/mL of imipridone derivative and about 100 mg/mL of nicotinamide.

The imipridone derivative or the pharmaceutically acceptable salt thereof is at least partially, preferably totally, dissolved in the liquid pharmaceutical composition. In some embodiments, the dissolved imipridone derivative or the pharmaceutically acceptable salt thereof in the liquid pharmaceutical composition represents at least 5% w/w of the liquid pharmaceutical composition.

The liquid aqueous solvent of the pharmaceutical composition preferably comprises water. Water may represent at least 20%, at least 30%, at least 40%, at least 50%, at least 60%, at least 70%, at least 80%, at least 90%, at least 50% or at least 99% v/v of the aqueous solvent. More preferably, the aqueous solvent is water. In other preferred embodiments, the aqueous solvent is a mixture of water and of one mono-alcohol, such as ethanol. In a specific embodiment, the aqueous solvent is a 1:1 water:ethanol mixture. In some embodiments, the aqueous solvent comprises at least 50 wt % of water.

The liquid pharmaceutical composition is preferably clear and transparent. The liquid pharmaceutical composition is preferably monophasic. In particular, it is not an emulsion nor a microemulsion.

The liquid pharmaceutical composition may further comprise at least one pharmaceutically acceptable pH modifier. The at least one pharmaceutically acceptable pH modifier preferably allows obtaining a pH of the composition comprised between 3 and 6. Thus, the pH of the liquid pharmaceutical composition is preferably comprised between 3 and 6, in particular about 4. The pH modifier may be selected from the group consisting of acetic acid, adipic acid, ammonium carbonate, ammonium hydroxide, ammonium phosphate, citric acid, diethanolamine, fumaric acid, hydrochloric acid, malic acid, nitric acid, propionic acid, potassium acetate, potassium bicarbonate, potassium chloride, potassium citrate, potassium metaphosphate, potassium phosphate, sodium acetate, sodium bicarbonate, sodium carbonate, sodium citrate, sodium glycolate, sodium hydroxide, sodium lactate, sodium phosphate, sodium propionate, succinic acid, sulfuric acid, tartaric acid, triethanolamine, arginine, lysine and meglumine. It is understood that pH adjustments may, after the addition of said pharmaceutically acceptable pH-modifying agent, be made by adding sufficient strong bases such as NaOH or KOH to neutralize to some extent the acid functions present, or in the opposite case, adding sufficient strong acids such as HCl, H₃PO₄ or H₂SO₄ to neutralize to some extent the basic functions present, so as to lead to the desired pH. The desired pH in the range of 3 to 6 helps increasing both the solubility of the imipridone derivative and its chemical stability.

The liquid pharmaceutical composition may further comprise at least one of:
an isotonizing agent,
a thickening and/or gelling agent,
a preservative, such as an antimicrobial agent and/or an antioxidant agent,
a flavoring agent,
a sweetening agent, and/or
a surfactant, preferably a non-ionic surfactant.

All these additional constituents are pharmaceutically acceptable. One skilled in the art is able to adapt the nature and/or amount of each additional constituent depending among others on the intended administration route, the amount of imipridone derivative, and/or the intended patient type (age or other).

The liquid pharmaceutical composition may further comprise any other suitable conventional additional constituent, especially if its addition to the composition does not substantially affect the therapeutical efficacy and/or the stability of the composition.

The isotonizing agent may be selected from the group consisting of a polyol, a sugar and a linear or cyclic glucitol having from 2 to 10 carbon atoms, for instance selected from mannitol, sorbitol, inositol, glucose and glycerol. This isotonizing agent can be chosen from sodium chloride, glucose and mannitol.

The thickening and/or gelling agent may be selected from the group consisting of polyvinyl alcohol, gelatin, cellulose derivatives, such as sodium carboxymethyl cellulose or hypromellose (hydroxypropylmethylcellulose HPMC, ethoxymethylcellulose, ester cellulose), povidone derivatives such as polyvinyl pyrrolidone; copovidone or povidone acetate, alginate derivatives, starch such as corn starch and gums such as guar gum, xanthan gum and/or carrageenan gum.

The preservative may be any preservative known in the art. It may be for instance selected from antimicrobial agents and antioxidant agents. The antimicrobial agent may be selected from the group consisting of sorbic acids, potassium sorbate, benzoates such as sodium benzoate, and parabens (alkyl parahydroxybenzoates). The antioxidant agent may be selected from the group consisting of ascorbic acid, erythorbic acid, monothioglycerol, sodium bisulfite, sodium metabisulfite, N-acetylcysteine, and a reducing sugar.

The sweetening agent may be selected from the group consisting of sucrose, sucralose, trehalose, saccharine, sodium saccharine, aspartame, acesulfame, stevia, maltodextrin and neohesperidin dihydrochalcone.

The flavoring agent or aroma may be selected from the group consisting of menthol, citrus, cola, strawberry, orange, mint and pepper mint.

The surfactant is preferably a non-ionic surfactant, and may be selected from the group consisting of polyoxyethylene hydrogenated castor oil (Kolliphor® RH 40), polysorbate 20 and 80 (Tween® 20-80) and caprylocaproyl polyoxylglycerides (Labrasol®). In some embodiments, at least one of the surfactants, preferably all surfactants, is (are) a hydrophilic surfactant(s).

The liquid pharmaceutical composition preferably comprises less than 5% w/v surfactants.

In an embodiment, the liquid pharmaceutical composition comprises no organic solvent. In some embodiments, the liquid aqueous composition may comprise at least one organic solvent, such ethanol, propylene glycol, polyethylene glycol (400-1000) or glycerol. The liquid pharmaceutical composition preferably comprises less than 50% v/v of a single mono-alcoholic solvent.

The liquid pharmaceutical composition may be viscous, for example when it comprises at least one thickening agent. The viscosity of the liquid pharmaceutical composition is preferably not higher than 40 000 cP at 20° C. Preferably, the viscosity of the liquid pharmaceutical composition is preferably not higher than 15 000 cP at 20° C. The viscosity may be measured at 20° C. by any suitable technique in the art, preferably with cone-plate rheometer such as, for instance RM200 CP4000 Plus.

The density of the liquid pharmaceutical composition is preferably not higher than 1.35. Preferably, the density of the liquid pharmaceutical composition is not higher than 1.3. In some embodiments, the density of the liquid pharmaceutical composition is not higher than 1.2, preferably not higher than 1.1. In some embodiments, the density of the liquid pharmaceutical composition is 1.0. The density may be measured by any suitable technique in the art, preferably with by weighing a precise volume of liquid using a scale thermostated at 20° C.

The liquid pharmaceutical composition is preferably suitable for injectable and/or oral administration. In an embodiment, the liquid pharmaceutical composition is an oral liquid pharmaceutical composition.

The liquid pharmaceutical composition may be sterilized by sterilizing filtration, aseptic distribution and/or autoclaving without affecting its stability nor its therapeutical efficacy.

The liquid pharmaceutical composition may be packaged in single-dose or multi-doses systems. Examples of single-dose systems suitable for liquid pharmaceutical compositions include vials, ampoules, sachets and sticks, such as four edge sealed bags and foil stick packs. Examples of multi-doses systems include bottles and vials.

Single-dose systems may have a volume comprised between 2 mL and 7 mL, preferably between 2 and 5.5 mL, in particular about 4 mL. Single-dose systems may each comprise between 200 and 625 mg of imipridone derivative, preferably between 200 and 550 mg of imipridone derivative, in particular about 400 mg of imipridone derivative.

### Complex of Imipridone Derivative and Nicotinamide

Another object of the present invention is a complex comprising at least one molecule of imipridone derivative as defined above and at least one molecule of nicotinamide. In a preferred embodiment, the complex comprises one molecule of imipridone derivative and one molecule of nicotinamide. Said preferred complex is a 1:1 imipridone derivative nicotinamide complex.

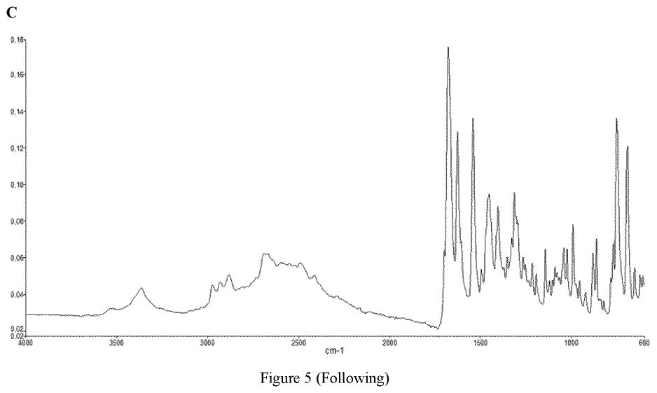

Preferably, the imipridone derivative is ONC201, more preferably ONC201.2HCl, and/or the complex preferably presents an infrared spectrum similar to that presented on FIG. 5 (a).

The complex comprising at least one molecule of imipridone derivative as defined above and at least one molecule of nicotinamide may be in the form of a cocrystal, preferably a cocrystal of one molecule of imipridone derivative and one molecule of nicotinamide.

Thus, another object of the present invention is a 1:1 imipridone derivative nicotinamide cocrystal. The imipridone derivative is preferably ONC201, more preferably ONC201.2HCl. The cocrystal is a new and distinct chemical composition of imipridone derivative (active agent) and nicotinamide (coformer). Cocrystals generally possess distinct crystallographic and spectroscopic properties when compared to those of the active agent and the coformer individually. Cocrystals often also exhibit distinct thermal behavior. Thermal behavior is measured by such techniques as capillary melting point, thermogravimetric analysis (TGA) and differential scanning calorimetry (DSC).

The complex and/or the cocrystal may be obtained by any suitable technique known in the art. For instance, they may be obtained, in solution, by the process for manufacturing a liquid composition according to the invention as described below. The process for preparing the complex and/or the cocrystal may further comprise a step of drying the solvent, and/or a step of co-crystallization, in particular in the solvent.

In some embodiments, the complex and/or the cocrystal may be obtained by the following process: the imipridone derivative and nicotinamide are first solubilized in water, preferably at a molar ratio of 0.98-1.02, imipridone derivative/nicotinamide. The water is then evaporated up to a loss of aqueous mass of at least 90% of its initially added mass, and the residue is recrystallized, for instance by gradual addition of isopropanol.

### Process for Manufacturing Liquid Compositions

Another object of the present invention is a process for manufacturing a liquid pharmaceutical composition according to the invention. Said process comprises the steps of:
(a) Dissolving nicotinamide in an aqueous solvent, preferably water;
(b) Adding at least one pH modifier and at least one imipridone derivative to the solution obtained in step (a); and
(c) Optionally sterilizing the solution obtained in step (b).

Step (a) may be performed by any suitable technique known in the art.

Step (b) is preferably performed under stirring. In some embodiments, the at least one pH modifier and the at least one imipridone derivative are added simultaneously to the solution; in other embodiments, the pH modifier is added before the imipridone derivative; in even other embodiments, the pH modifier is added after the imipridone derivative. Both additions may be separated by a stirring period. The addition of the imipridone derivative is preferably performed at a temperature comprised between 2° and 45° C., more preferably between 2° and 25° C. Alternatively, the obtained solution may be stirred at a temperature comprised between 2° and 45° C., more preferably between 2° and 25° C. after the imipridone derivative is added.

The process may further comprise, between step (b) and step (c), a step (b′) of homogenizing the solution obtained in step (b). Said homogenizing may be performed under stirring, preferably until a clear and transparent solution is obtained.

Step (c) may be performed by any suitable technique known in the art. The suitable technique may be selected depending of the route of administration intended for the pharmaceutical composition. For instance, step (c) may be performed by autoclaving, by aseptic distribution or by filtration, for instance by filtration on a 0.22 μm filter.

Steps (a) to(s) are preferably implemented in this order. Nevertheless, in specific cases, the order may be changed depending on the parameters of the process or of the composition to obtain.

### Solid Composition

Another object of the invention is a solid pharmaceutical composition comprising at least one imipridone derivative, or a pharmaceutically acceptable salt and/or solvate thereof and nicotinamide, which is obtained from a liquid composition comprising an aqueous solvent, the at least one imipridone derivative, or a pharmaceutically acceptable salt and/or solvate thereof and nicotinamide. The imipridone derivative, or the pharmaceutically acceptable salt and/or solvate thereof is at least partially dissolved, preferably totally dissolved, in the liquid composition thanks to the presence of nicotinamide. The liquid composition is preferably a liquid pharmaceutical composition according to the invention as described above.

In the solid pharmaceutical composition, the imipridone derivative and nicotinamide are preferably in the form of a complex or a cocrystal, preferably a 1:1 complex or a 1:1 cocrystal. The complexation of the imipridone derivative and nicotinamide is believed to increase, among others, the bioavailability of the imipridone derivative after its administration to a subject.

The solid pharmaceutical composition may be obtained by gelling the liquid aqueous composition, by spray drying the liquid aqueous composition, by encapsulating the liquid composition and/or by evaporating at least part of the aqueous solvent of the liquid composition.

The invention also relates to a process for manufacturing a solid pharmaceutical composition according to the invention, comprising the following steps:
(i) Providing a liquid composition comprising an aqueous solvent, at least one imipridone derivative, or a pharmaceutically acceptable salt and/or solvate thereof and nicotinamide;
(ii) Transforming at least part of the liquid composition provided in step (a) into a solid composition, preferably by gelling the liquid aqueous composition, by encapsulating the liquid composition and/or by evaporating at least part of the aqueous solvent of the liquid composition.

The solid pharmaceutical composition may be in any suitable solid form. The solid composition form may be selected from the group consisting of gummies, dragees, powders for reconstitution in water, orodispersible films and capsules, such as hard-gel capsules and/or soft-gel capsules, preferably soft-gel capsules.

When the solid composition form is a gummy, step (ii) of the above process may be implemented by a deposition, drying and molding process.

When the solid composition form is an orodispersible film, step (ii) of the above process may be implemented by a deposition and drying process, such as 3D-printing.

When the solid composition form is a capsule, step (ii) of the above process may be implemented by a filling process.

When the solid composition form is a hard-gel capsule, the density of the liquid composition is preferably quite high, for instance up to 1.35, and/or the viscosity of the liquid composition is preferably quite high, for instance up to 40 000 cP, the viscosity and/or density being measured as detailed above.

The solid pharmaceutical composition is preferably suitable for oral administration. In an embodiment, the solid pharmaceutical composition is an oral solid pharmaceutical composition.

The solid pharmaceutical composition may be a single-dose or a multi-dose composition.

### Composition for Use

Another object of the invention is a pharmaceutical composition according to the invention, which is a liquid pharmaceutical composition or a solid pharmaceutical composition according to the invention, for use as a medicament.

Another object of the invention is a pharmaceutical composition according to the invention, which is a liquid pharmaceutical composition or a solid pharmaceutical composition according to the invention, for use in the treatment of cancer in a subject. The cancer to be treated according to the invention may be selected from the group consisting of prostate carcinoma, pancreatic cancer, lung carcinoma, breast cancer, glioma, cancers of head and neck, colon cancer and skin cancer.

It is preferably a glioma, more preferably a diffuse midline glioma such as H3K28 altered diffuse midline glioma.

The dose of imipridone derivative in the composition for use according to the invention may be any dose suitable for treating the pathology and/or condition. For instance, the dose of imipridone derivative may range between 50 and 500 mg/m², preferably between 350 and 400 mg/m², in particular about 375 mg/m². When the composition for use is a liquid composition for use, the concentration of imipridone derivative in the liquid composition may range from 10 to 200 mg/mL, preferably between 100 and 200 mg/mL. the volume of the dose of liquid composition to be administered according to the invention is preferably not higher than 5 mL, especially when the patient is a child.

The pharmaceutical composition for use according to the invention may be co-administered with any other suitable treatment, in particular with any other suitable anti-cancer treatment. The co-administration may be simultaneous, sequential or separate.

The invention also relates to the use of an imipridone derivative and nicotinamide, or of a pharmaceutical composition comprising an imipridone derivative and nicotinamide, for the manufacture of a medicament.

The invention further relates to a method for treating a disease or disorder in a subject in need thereof, comprising administering a therapeutically effective amount of an imipridone derivative in combination with nicotinamide, or a pharmaceutical composition comprising a therapeutically effective amount of an imipridone derivative and nicotinamide, to said subject. The disease or disorder is preferably a cancer as described above.

The invention will also be described in further detail in the following examples, which are not intended to limit the scope of this invention, as defined by the attached claims.

### EXAMPLES

### Example 1: Formulation of an Orodispersible Film According to the Invention

TABLE 1

| | UNIT | RELATIVE |
|---|---|---|
| | COMPOSITION | COMPOSITION |
| INGREDIENTS | (mg) | (%) |
| ONC 201.2HCl | 59 mg | 5.9% |
| Nicotinamide | 50 mg | 5.0% |
| HPMC | 75 mg | 7.5% |
| Sucralose | 2.5 mg | 0.25% |
| Mint | 1 mg | 0.1% |
| Solvent system | Qs 1000 mg | Qs 100% |
| (water:ethanol | | |
| 1:1 w:w) | | |

### Example 2: Formulation of Gummies According to the Invention

TABLE 2

| | UNIT COMPOSITION | | RELATIVE |
|---|---|---|---|
| INGREDIENTS | (mg) | | COMPOSITION (%) |
| ONC 201.2HCl | 60 | mg | 8.4% |
| Nicotinamide | 50 | mg | 7.0% |
| Corn starch | 31 | mg | 4.4% |
| Carrageenan gum | 12 | mg | 1.7% |
| Gelatin | 50 | mg | 7.0% |
| Xanthan gum | 2 | mg | 0.3% |
| Sucralose | 5 | mg | 0.7% |
| Mint aroma | 1.2 | mg | 0.2% |
| Water | 500 | mg | 70.3% |
| Total | 711.2 | mg | 100.0% |

### Example 3: Formulation of Gummies According to the Invention

TABLE 3

| | UNIT COMPOSITION | | RELATIVE |
|---|---|---|---|
| INGREDIENTS | (mg) | | COMPOSITION (%) |
| ONC 201.2HCl | 60 | mg | 6.1% |
| Nicotinamide (already | 50 | mg | 5.1% |
| dissolved) | | | |
| HPMC | 13.5 | mg | 1.4% |
| Corn starch | 286 | mg | 29.2% |
| Gelatin | 68 | mg | 6.9% |
| Sucralose | 2.7 | mg | 0.3% |
| Mint | 0.6 | mg | 0.06% |
| Water | 500 | mg | 51.0% |
| Total | 980.8 | mg | 100.0% |

### Example 4. Resistance to Oxidation

An aqueous solution of 100 mg/mL of ONC201.2HCl and 100 mg/mL nicotinamide (1:1 weight ratio) was subjected to oxidative stress in the presence of hydrogen peroxide at 0.3% v/v. The same experiment was performed with a comparative solution of 100 mg/mL of ONC201.2HCl, in the absence of nicotinamide.

A clear solution was obtained for both solutions at the beginning of the experiment.

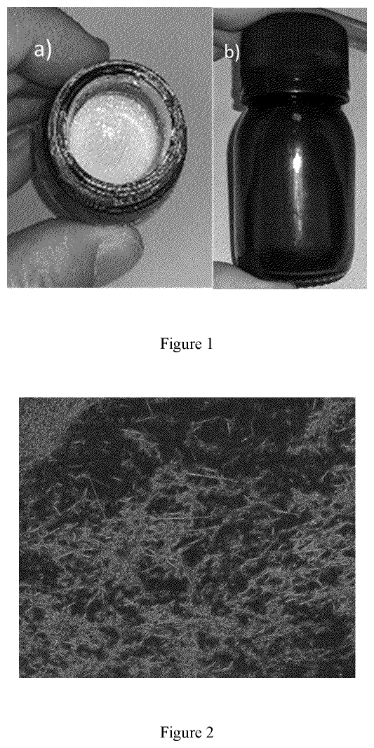

A substantial loss of ONC201.2HCl was rapidly observed for the comparative solution that does not comprise nicotinamide, concomitantly with a detection of numerous oxidation products. The degradation products may be detected for instance by liquid chromatography-high-resolution multistage mass spectrometry. After 3 days of storage at room temperature, the comparative solution looked opaque. FIG. 1 presents the appearance of the solution after 3 days of storage at room temperature (a), and at the beginning of the experiment (b). FIG. 2 presents a microscopy image of the mixture of FIG. 1 (a), observed at 20× magnification. Acicular crystals are observed.

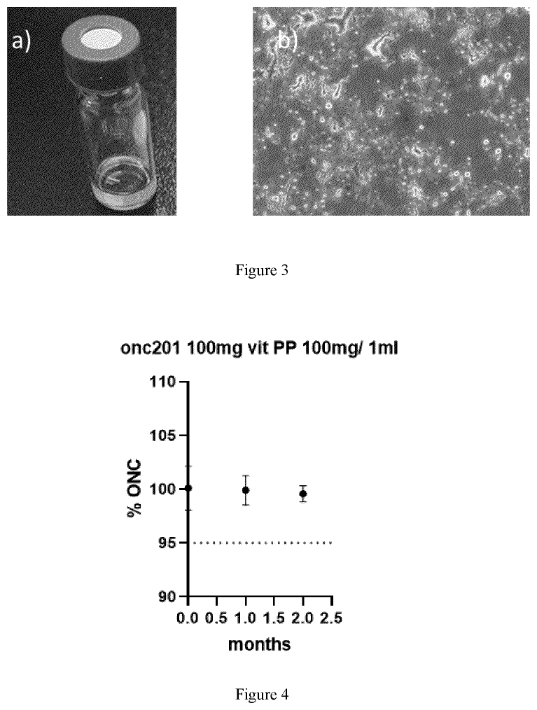

Conversely, the solution comprising nicotinamide remained clear after 5 days of storage at room temperature. FIG. 3 presents the appearance of the solution after 5 days of storage at room temperature (a), and a microscopy image of this mixture, observed at 20× magnification. Small non-crystalline clusters are present. No acicular crystals are observed.

### Example 5: Stability of a Liquid Solution Under Storage

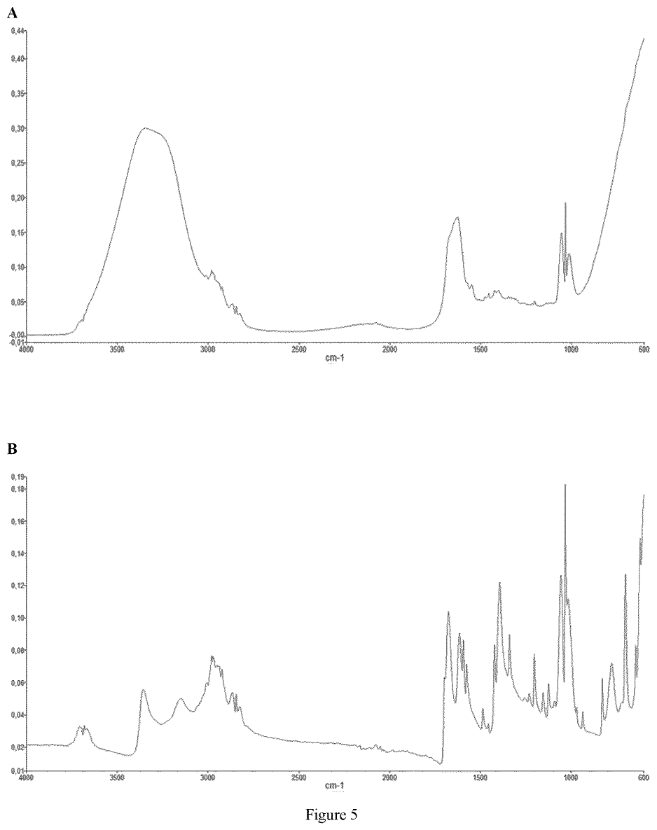

An aqueous solution of 100 mg/mL of ONC201.2HCl and 100 mg/mL nicotinamide (1:1 weight ratio) was stored at room temperature for 2.5 months. FIG. 4 presents the evolution of the amount of imipridone derivative over time in the solution. Such amount remains globally constant over 2.5 months.

### Example 6: Preparation and Characterization of a 1:1 Complex According to the Invention

A liquid composition of nicotinamide and ONC201.2HCl was prepared as follows: ONC201.2HCl and nicotinamide are first solubilized in water in a 1:1 mass ratio, ONC201.2HCl:nicotinamide. The water is then evaporated to a loss of the aqueous mass of at least 90% of its initially added mass and the spectra of the resulting ternary mixture were recorded by ATR-IR.

The obtained liquid composition was characterized by infrared (IR) spectroscopy. The IR absorption spectrum of the mixture is presented in FIG. 5a). FIGS. 5b) and 5c) respectively present the IR spectra of nicotinamide alone and of ONC201.2HCl alone.

A small amount of residual water can be seen on the spectrum of FIG. 5a). The disappearance of some absorption bands on the spectrum of the liquid composition comprising nicotinamide and ONC201.2HCl when compared to the spectra of individual components clearly suggests an interaction exists between both compounds. This proves the complexation between nicotinamide and the imipridone derivative.

### Example 7. Extrapolation to ONC206 and ONC212

By leveraging quantum molecular descriptors linked to solubility and stability, the inventors aim to demonstrate the scientific rationale for extending solubility and stability experimental results from ONC201 to its structurally related counterparts ONC206 and ONC212.

Highlighting the foundational similarities between ONC201, ONC206, and ONC212 (and their hydrochloride salts), this report establishes a robust scientific basis for extrapolating findings from ONC201 to ONC206 and ONC212, based on predictive molecular modeling.

### Methodological Approach:

The inventors employed advanced computational methodologies, using Jaguar and Maestro programs from the Schrödinger Suite 2019-1, to analyze quantum molecular descriptors indicative of solubility and stability. This approach ensures high accuracy and reliability in comparing ONC201, ONC206, and ONC212. Selection of the B3LYP exchange-correlation functional with the D3 a posteriori correction and the 6-31G++** basis set was driven by their proven effectiveness in accurately modeling molecular behaviors critical to solubility and stability [1,2]. pKa were calculated using the Epik suite included in Maestro Software. Log P and log D at pH 5 were obtained using the online calculator plugin proposed by Chemaxon.

### REFERENCES

[1] A. D. Bochevarov, E. Harder, T. F. Hughes, J. R. Greenwood, D. A. Braden, D. M. Philipp, et al., Jaguar: a high-performance quantum chemistry software program with strengths in life and materials sciences, Int. J. Quantum Chem. 113 (2013) 2110-2142.
[2] K. Theilacker, A. V. Arbuznikov, H. Bahmann, M. Kaupp, Evaluation of a combination of local hybrid functionals with DFT-D3 corrections for the calculation of thermochemical and kinetic data, J. Phys. Chem. A 115 (2011) 8990-8996.

### Anticipated Findings and Significance

Our computational strategy is tailored to generate convincing data that elucidate the similarities in solubility and stability between ONC201 and its analogs, ONC206 and ONC212.

The subsequent table details the comparative analysis of key quantum molecular descriptors across the three compounds.

TABLE 4

| Descriptor | ONC201 | ONC206 | ONC212 | Description |
|---|---|---|---|---|
| pKa | 7.02; 8.86 | 7.02; 8.86 | 7.02; 8.86 | Acid dissociation constant, |
| | | | | influencing solubility and stability in |
| | | | | different pH environments. |
| logP | 3.2 | 3.1 | 3.5 | Partition coefficient, indicating |
| | | | | lipophilicity and impacts on |
| | | | | membrane permeability and |
| | | | | solubility. |
| logD at pH 5 | 1.5 | 1.8 | 2.1 | Distribution coefficient at pH 5, |
| | | | | offering insight into solubility and |
| | | | | permeability at physiological pH. |
| ALIE at pH 5 | 206.95; | 213.74; | 213.59; | Minimal, mean and maximal Average |
| | 279.48; | 288.38; | 290.23; | Local Ionization Energy (ALIE) in |
| | 401.27 | 400.71 | 401.15 | kcal/mol at pH 5. Related to |
| | | | | molecular reactivity and stability. |
| MEP at pH 5 | 66.66; | 51.69; | 45.58; | Minimal, mean and maximal |
| | 126.57; | 128.85; | 126.70; | Molecular Electrostatic Potential |
| | 218.74 | 221.24 | 223.03 | (MEP) in kcal/mol at pH 5, |
| | | | | highlighting interaction sites and |
| | | | | potential reactivity. |
| HOMO at pH 5 | −6.942 | −7.263 | −7.285 | Energy of the Highest Occupied |
| (eV) | | | | Molecular Orbital (HOMO) at pH 5, a |
| | | | | marker of chemical stability and |
| | | | | reactivity. |
| LUMO at pH 5 | −2.119 | −2.147 | −2.179 | Energy of the Lowest Unoccupied |
| (eV) | | | | Molecular Orbital (LUMO) at pH 5, |
| | | | | indicative of the molecule's |
| | | | | ability to accept electrons. |
| HOMO-LUMO | 4.255 | 4.327 | 4.385 | Difference in energy between HOMO |
| Energy Gap (eV) | | | | and LUMO, relevant for chemical |
| | | | | stability and reactivity. |
| Chemical | −4.823 | −5.116 | −5.107 | A measure of resistance to change in |
| Hardness (eV) | | | | electron distribution, related to |
| | | | | stability. |
| Chemical | 2.412 | 2.558 | 2.553 | Tendency of a molecule to gain or |
| Potential (eV) | | | | lose electrons, impacting reactivity. |
| Electrophilicity | 4.530 | 4.705 | 4.732 | Likelihood of being an electron |
| (eV) | | | | acceptor, influencing solubility |
| | | | | and reactivity. |

1-21. (canceled)

22. A liquid pharmaceutical composition comprising an aqueous solvent, nicotinamide and at least one imipridone derivative of formula (I): 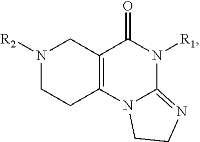
wherein R₁ is an alkyl group substituted by a heterocycle or a substituted benzyl group and R₂ is a hydrogen atom or a benzyl group, or a pharmaceutically acceptable salt and/or solvate thereof.

23. The liquid pharmaceutical composition according to claim 22, wherein the imipridone derivative of formula (I) is selected from the group consisting of ONC201, ONC206 and ONC212 or a pharmaceutically acceptable salt and/or solvate thereof or is ONC201.2HCl.

24. The liquid pharmaceutical composition according to claim 22, wherein the aqueous solvent is water or a 1:1 w:w water:ethanol mixture.

25. The liquid pharmaceutical composition according to claim 22, wherein the composition further comprises at least one pharmaceutically acceptable pH modifier.

26. The liquid pharmaceutical composition according to claim 22, wherein the composition further comprises at least one of
an isotonizing agent,
a thickening and/or gelling agent,
a preservative, antimicrobial agent and/or an antioxidant agent,
a flavoring agent,
a sweetening agent, and/or
a surfactant.

27. The liquid pharmaceutical composition according to claim 26, wherein
the isotonizing agent is selected from the group consisting of a polyol, a sugar and a linear or cyclic glucitol having from 2 to 10 carbon atoms; and/or
the thickening and/or gelling agent is selected from the group consisting of polyvinyl alcohol, gelatin, cellulose derivatives, povidone derivatives; copovidone or povidone acetate, alginate derivatives, starch and gums; and/or
the antibacterial agent is selected from the group consisting of sorbic acids, potassium sorbate, benzoates, and parabens; and/or
the antioxidant agent is selected from the group consisting of ascorbic acid, erythorbic acid, monothioglycerol, sodium bisulfite, sodium metabisulfite, N-acetylcysteine, and a reducing sugar; and/or
the sweetening agent is selected from the group consisting of sucrose, sucralose, trehalose, saccharine, sodium saccharine, aspartame, acesulfame, stevia, maltodextrin and neohesperidin dihydrochalcone; and/or
the flavoring agent is selected from the group consisting of menthol, citrus, cola, strawberry, orange, mint and peppermint; and/or
the surfactant is selected from the group consisting of polyoxyethylene hydrogenated castor oil, polysorbate 20, polysorbate 80 and caprylocaproyl polyoxylglycerides.

28. The liquid pharmaceutical composition according to claim 22, wherein the weight ratio of imipridone derivative to nicotinamide is between 0.5 and 2.

29. The liquid pharmaceutical composition according to claim 22, wherein the amount of imipridone derivative is between 20 mg/mL and 420 mg/mL.

30. The liquid pharmaceutical composition according to claim 22, wherein the amount of nicotinamide is between 20 mg/mL and 420 mg/mL.

31. A process for manufacturing a liquid pharmaceutical composition according to claim 22, wherein the process comprises the steps of:
(a) dissolving nicotinamide in an aqueous solvent;
(b) adding at least one pH modifier and at least one imipridone derivative to the solution obtained in step (a); and
(c) optionally sterilizing the solution obtained in step (b).

32. A solid pharmaceutical composition comprising at least one imipridone derivative, or a pharmaceutically acceptable salt and/or solvate thereof and nicotinamide, which is obtained from a liquid aqueous composition according to claim 22.

33. The solid pharmaceutical composition according to claim 32, wherein the form of the solid pharmaceutical composition is selected from the group consisting of gummies, dragees, powders for reconstitution in water, orodispersible films, hard-gel capsules and soft-gel capsules.

34. A 1:1 imipridone derivative nicotinamide complex or cocrystal, wherein the imipridone derivative is of formula (I): 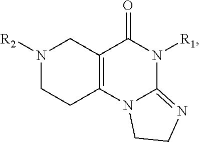
wherein R₁ is an alkyl group substituted by a heterocycle or a substituted benzyl group and R₂ is a hydrogen atom or a benzyl group; ONC201; or ONC201.2HCl.

35. A method for treating a disease or disorder in a subject in need thereof, comprising administering a therapeutically effective amount of a liquid pharmaceutical composition according to claim 22 to said subject.

36. The method according to claim 35, wherein the disease or disorder is a cancer.

37. The method according to claim 36, wherein the cancer is selected from the group consisting of prostate carcinoma, pancreatic cancer, lung carcinoma, breast cancer, glioma, diffuse midline glioma, cancers of head and neck, colon cancer and skin cancer.

38. A method for treating a disease or disorder in a subject in need thereof, comprising administering a therapeutically effective amount of a solid pharmaceutical composition according to claim 32 to said subject.

39. The method according to claim 38, wherein the disease or disorder is a cancer.

40. The method according to claim 39, wherein the cancer is selected from the group consisting of prostate carcinoma, pancreatic cancer, lung carcinoma, breast cancer, glioma, diffuse midline glioma, cancers of head and neck, colon cancer and skin cancer.
